(19) United States
(12) Patent Application Publication
(10) Pub. No.: US 2007/0073133 A1
(43) Pub. Date: Mar. 29, 2007

(54) Virtual mouse for use in surgical navigation

(76) Inventor: Ryan J. Schoenefeld, Fort Wayne, IN (US)

Correspondence Address: Intellectual Property Group;Bose McKinney & Evans LLP, 2700 First Indiana Plaza, 135 North Pennsylvania Street, Indianapolis, IN, 46204, US

(21) Appl. No.: 11/227,741

(22) Filed: Sep. 15, 2005

(51) Int. Cl. A61B 5/05

(52) U.S. Cl. 600/407

(57) ABSTRACT

A surgical navigation system including a computer, a tracking system, and patient anatomical information. The surgical navigation system includes a structure based control system to enable a surgeon to reduce surgery time and costs. A virtual mouse or its functional equivalent is provided to enable a surgeon to access various features of the software based control system.

### BACKGROUND

The present invention relates generally to image guided surgery and more particularly to a method of using a computer in an image guided surgery procedure.

Surgical navigation systems, also known as computer assisted surgery and image guided surgery, aid surgeons in locating patient anatomical structures, guiding surgical instruments, and implanting medical devices with a high degree of accuracy. Surgical navigation has been compared to a global positioning system that aids vehicle operators to navigate the earth. A surgical navigation system typically includes a computer, a tracking system, and patient anatomical information. The patient anatomical information can be obtained by using an imaging mode such a fluoroscopy, computer tomography (CT) or by simply defining the location of patient anatomy with the surgical navigation system. Surgical navigation systems can be used for a wide variety of surgeries to improve patient outcomes.

To successfully implant a medical device, surgical navigation systems often employ various forms of computing technology, as well as utilize intelligent instruments, digital touch devices, and advanced 3-D visualization software programs. All of these components enable surgeons to perform a wide variety of standard and minimally invasive surgical procedures and techniques. Moreover, these systems allow surgeons to more accurately plan, track and navigate the placement of instruments and implants relative to a patient's body, as well as conduct pre-operative and intra-operative body imaging.

To accomplish the accurate planning, tracking and navigation of surgical instruments, tools and/or medical devices during an image guided surgery procedure, surgeons often utilize “tracking arrays” that are coupled to the surgical components. The tracking arrays allow the surgeon to accurately track the location of these surgical components, as well as the patient's bones during the surgery. By knowing the physical location of the tracking array, the software detection program of the tracking system is able to calculate the position of the tracked component relative to a surgical plan image.

It is known to employ a keypad on the back of a universal calibrator used in image guided surgery. This “virtual keypad” allows the user to access certain system functions from the sterile field without using the touch screen or mouse, the latter items being located outside of the sterile field. The enabled functions of known virtual keypads vary depending on application, but are accessed in the same manner. The user touches the desired button on the virtual keypad using the tip of a calibrated probe (or calibrated drill guide). The array of the universal calibrator and the probe array (or drill guide array) must be in view of the camera to enable the virtual keypad function.

The known virtual keypad is limited in the number of tasks that are pre-programmed into the software.

### SUMMARY OF THE INVENTION

The present teachings provide an apparatus and method for using a probe or other surgical instrument that is tracked during a surgical procedure as a virtual mouse or its functional equivalent.

In one form thereof, there is provided a method of performing a surgery. This method includes operating a surgical navigation system having a tracking system, computer and monitor that are placed outside of a sterile field. A pad having a pad array and a probe having a probe array are placed within the sterile field. The pad array and probe array are acquired with the tracking system. The virtual mouse is activated by moving the probe near the pad, and a mouse input to the computer is made with the virtual mouse.

In exemplary embodiments, the mouse input comprises moving a pointer on the monitor. This is typically accomplished by moving the probe along a substantially flat surface of the pad. In other exemplary embodiments, the probe is moved away from the surface of the pad to make a second mouse input to the computer. This second input could be interpreted by the computer as the equivalent of a single click of a conventional mouse. It may also be interpreted as a double click, scrolling the monitor or other mouse inputs. In yet other exemplary embodiments, the probe is moved away from the pad and further movement of the probe in three dimensions correspondingly manipulates an object on the computer monitor. The object may be a human anatomy image.

### BRIEF DESCRIPTION OF DRAWINGS

The above-mentioned aspects of the present teachings and the manner of obtaining them will become more apparent and the invention itself will be better understood by reference to the following description of the embodiments of the invention taken in conjunction with the accompanying drawings, wherein:

FIG. 1 is a perspective view of an operating room setup in a computer assisted surgery in accordance with an embodiment of the present invention;

FIG. 2 is an exemplary block diagram of a surgical navigation system embodiment in accordance with the present invention;

FIG. 3 is an exemplary surgical navigation kit embodiment in accordance with the present invention;

FIG. 4 is a flowchart illustrating the operation of an exemplary surgical navigation system in accordance with the present invention;

FIG. 5 shows a first exemplary computer display layout embodiment in accordance with the present invention;

FIG. 6 is a fragmentary perspective view illustrating a virtual mouse and a method of using the virtual mouse in accordance with the present invention;

FIG. 7 is a block diagram illustrating the activation of a virtual mouse in accordance with the present invention;

FIGS. 8-11 are fragmentary perspective views illustrating a virtual mouse and a method of using the virtual mouse in accordance with the present invention; and

FIG. 12 is a block diagram which describes various features of embodiments incorporating the present invention.

Corresponding reference characters indicate corresponding parts throughout the several views.

### DETAILED DESCRIPTION

The embodiments of the present invention described below are not intended to be exhaustive or to limit the invention to the precise forms disclosed in the following detailed description. Rather, the embodiments are chosen and described so that others skilled in the art may appreciate and understand the principles and practices of the present invention.

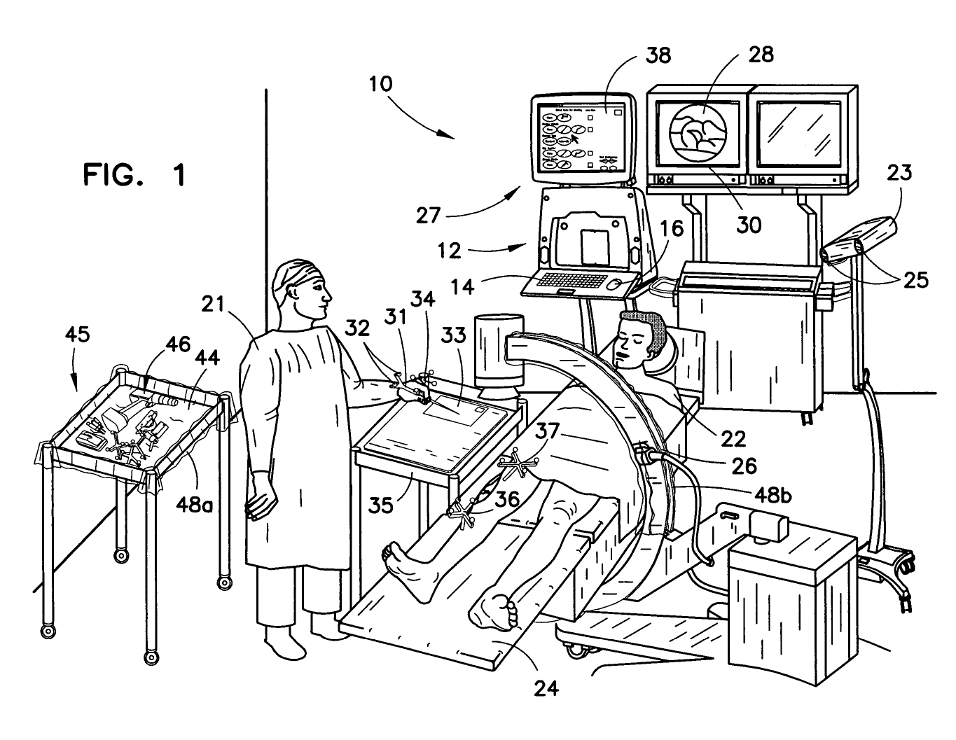

FIG. 1 shows a perspective view of an operating room with surgical navigation system 10. System 10 may include one or more computers 12 which may be operated by a keyboard 14 and a conventional or physical mouse 16, all of which may be located outside the sterile field. Physician or surgeon 21 is aided by the surgical navigation system in performing knee arthroplasty, also known as knee replacement surgery, on patient 22 shown lying on operating table 24. Surgical navigation system 10 has a tracking system that locates arrays and tracks them in real-time. To accomplish this, the surgical navigation system includes optical locator 23, which has two CCD (charge couple device) cameras 25 that detect the positions of the arrays in space by using triangulation methods. The relative location of the tracked arrays, including the patient's anatomy, can then be shown on a computer display (such as computer display 27 for instance) to assist the surgeon during the surgical procedure. The arrays that are typically used include probe arrays, instrument arrays, reference arrays, and calibrator arrays. The operating room includes an imaging system such as C-arm fluoroscope 26 with fluoroscope display image 28 to show a real-time image of the patient's knee on monitor 30. Physician 21 may use surgical probe 31 to reference a point on the patient's knee, and reference arrays 36 and 37 attached to the patient's femur and tibia to provide known anatomic reference points so the surgical navigation system can compensate for leg movement.

In addition, as illustrated here, physician 21 may use probe 31, having markers 32, as a virtual mouse in combination with a touch pad 33 and a locating array 34. The pad 33 and locating array 34 may be supported by a stand or table 35 or other suitable structure for support within reach of the surgeon 21. A display image or user interface screen 38 displayed on display 27 includes a plurality of icons for selection by the physician 21 through use of the virtual mouse. The virtual mouse is typically located within the sterile field.

The operating room also includes instrument cart 45 having tray 44 for holding a variety of surgical instruments and arrays 46. Instrument cart 45 and C-arm 26 are typically draped in sterile covers 48a, 48b to eliminate contamination risks within the sterile field.

The surgery is performed within the sterile field, adhering to the principles of asepsis by all scrubbed persons in the operating room. Patient 22 and physician 21 are prepared for the sterile field through appropriate scrubbing and clothing. The sterile field will typically extend from operating table 24 upward in the operating room. Typically both computer display image 38 and fluoroscope display image 28 are located outside of the sterile field.

A representation of the patient's anatomy can be acquired with an imaging system, a virtual image, a morphed image, or a combination of imaging techniques. The imaging system can be any system capable of producing images that represent the patient's anatomy such as a fluoroscope producing x-ray two-dimensional images, computer tomography (CT) producing a three-dimensional image, magnetic resonance imaging (MRI) producing a three-dimensional image, ultrasound imaging producing a two-dimensional image, and the like. A virtual image of the patient's anatomy can be created by defining anatomical points with surgical navigation system 10 or by applying a statistical anatomical model. A morphed image of the patient's anatomy can be created by combining an image of the patient's anatomy with a data set, such as a virtual image of the patent's anatomy. Some imaging systems, such as C-arm fluoroscope 26, may require calibration. The C-arm may be calibrated with a calibration grid that enables determination of fluoroscope projection parameters for different orientations of the C-arm to reduce distortion. A registration phantom may also be used with a C-arm to coordinate images with the surgical navigation application program and improve scaling through the registration of the C-arm with the surgical navigation system. A more detailed description of C-arm based navigation system is provided in James B. Stiehl et al., Navigation and Robotics in Total Joint and Spine Surgery, Chapter 3 C-Arm-Based Navigation, Springer-Verlag (2004).

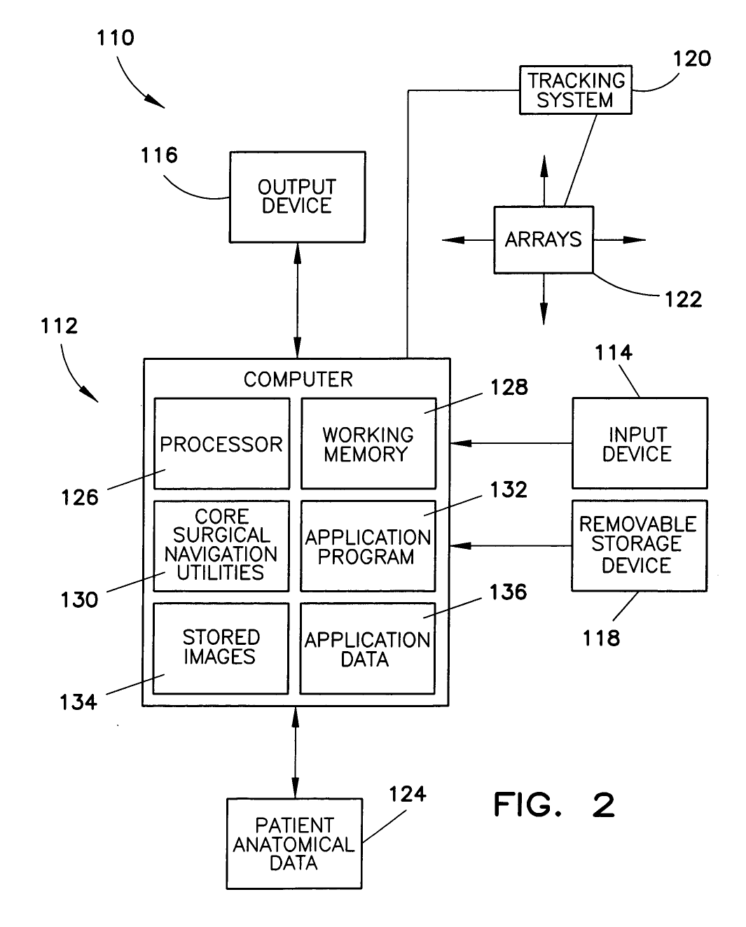

FIG. 2 is a block diagram of an exemplary surgical navigation system embodiment in accordance with the present teachings, such as an Acumen™ Surgical Navigation System available from EBI, L.P., Parsipanny, N.J. USA, a Biomet Company. The surgical navigation system 110 comprises computer 112, input device 114, output device 116, removable storage device 118, tracking system 120, arrays 122, and patient anatomical data 124, as further described in the brochure Acumen™ Surgical Navigation System, Understanding Surgical Navigation (2003), available from EBI, L.P. The Acumen™ Surgical Navigation System can operate in a variety of imaging modes such as a fluoroscopy mode creating a two-dimensional x-ray image, a computer-tomography (CT) mode creating a three-dimensional image, and an imageless mode creating a virtual image or planes and axes by defining anatomical points of the patient's anatomy. In the imageless mode, a separate imaging device such as a C-arm is not required, thereby simplifying set-up. The Acumen™ Surgical Navigation System may run a variety of orthopedic applications, including applications for knee arthroplasty, hip arthroplasty, spine surgery, and trauma surgery, as further described in the brochure “Acumen™ Surgical Navigation System, Surgical Navigation Applications” (2003) available from EBI, L.P. A more detailed description of an exemplary surgical navigation system is provided in James B. Stiehl et al., Navigation and Robotics in Total Joint and Spine Surgery, Chapter 1 Basics of Computer-Assisted Orthopedic Surgery (CAOS), Springer-Verlag (2004).

Computer 112 may be any computer capable of properly operating surgical navigation devices and software, such as a computer similar to a commercially available personal computer that comprises a processor 126, working memory 128, core surgical navigation utilities 130, an application program 132, stored images 134, and application data 136. Processor 126 is a processor of sufficient power for computer 112 to perform desired functions, such as one or more microprocessors. Working memory 128 is memory sufficient for computer 112 to perform desired functions such as solid-state memory, random-access memory, and the like. Core surgical navigation utilities 130 are the basic operating programs, and include image registration, image acquisition, location algorithms, orientation algorithms, virtual keypad, diagnostics, and the like. Application program 132 may be any program configured for a specific surgical navigation purpose, such as orthopedic application programs for unicondylar knee (“uni-kee”), total knee, hip, spine, trauma, intramedullary (“IM”) nail, and external fixator. Stored images 134 are those recorded during image acquisition using any of the imaging systems previously discussed. Application data 136 is data that is generated or used by application program 132, such as implant geometries, instrument geometries, surgical defaults, patient landmarks, and the like. Application data 136 can be pre-loaded in the software or input by the user during a surgical navigation procedure.

Output device 116 can be any device capable of creating an output useful for surgery, such as a visual output and an auditory output. The visual output device can be any device capable of creating a visual output useful for surgery, such as a two-dimensional image, a three-dimensional image, a holographic image, and the like. The visual output device can be a monitor for producing two and three-dimensional images, a projector for producing two and three-dimensional images, and indicator lights. The auditory output may be any device capable of creating an auditory output used for surgery, such as a speaker that may be used to provide a voice or tone output.

Removable storage device 118 may be any device having a removable storage media that would allow downloading data such as application data 136 and patient anatomical data 124. The removable storage device can be a read-write compact disc (CD) drive, a read-write digital video disc (DVD) drive, a flash solid-state memory port, a removable hard drive, a floppy disc drive, computer readable medium, and the like.

Tracking system 120 can be any system that can determine the three-dimensional location of devices carrying or incorporating markers that serve as tracking indicia. An active tracking system has a collection of infrared light emitting diode (ILEDs) illuminators that surround the position sensor lenses to flood a measurement field of view with infrared light. A passive system incorporates retro-reflective markers that reflect infrared light back to the position sensor, and the system triangulates the real-time position (x, y, and z location) and orientation (rotation around x, y, and z axes) of an array 122 and reports the result to the computer system with an accuracy of about 0.35 mm Root Mean Squared (RMS). An example of passive tracking system is a Polaris® Passive System and an example of a marker is the NDI Passive Spheres™ both available from Northern Digital Inc. Ontario, Canada. A hybrid tracking system can detect active and active wireless markers in addition to passive markers. Active marker based instruments enable automatic tool identification, program control of visible LEDs, and input via tool buttons. An example of a hybrid tracking system is the Polaris® Hybrid System available from Northern Digital Inc. A marker can be a passive IR reflector, an active IR emitter, an electromagnetic marker, and an optical marker used with an optical camera.

Arrays 122 can be probe arrays, instrument arrays, reference arrays, calibrator arrays, and the like. Arrays 122 can have any number of markers, but typically have three or more markers to define real-time position (x, y, and z location) and orientation (rotation around x, y, and z axes). As will be explained in greater detail below, an array comprises a body and markers. The body comprises an area for spatial separation of markers. In some embodiments, there are at least two arms and some embodiments can have three arms, four arms, or more. The arms are typically arranged asymmetrically to facilitate specific array and marker identification by the tracking system. In other embodiments, such as a calibrator array, the body provides sufficient area for spatial separation of markers without the need for arms. Arrays can be disposable or non-disposable. Disposable arrays are typically manufactured from plastic and include installed markers. Non-disposable arrays are manufactured from a material that can be sterilized, such as aluminum, stainless steel, and the like. The markers are removable, so they can be removed before sterilization.

Planning and collecting patient anatomical data 124 is a process by which a clinician inputs into the surgical navigation system actual or approximate anatomical data. Anatomical data can be obtained through techniques such as anatomic painting, bone morphing, CT data input, and other inputs, such as ultrasound and fluoroscope and other imaging systems.

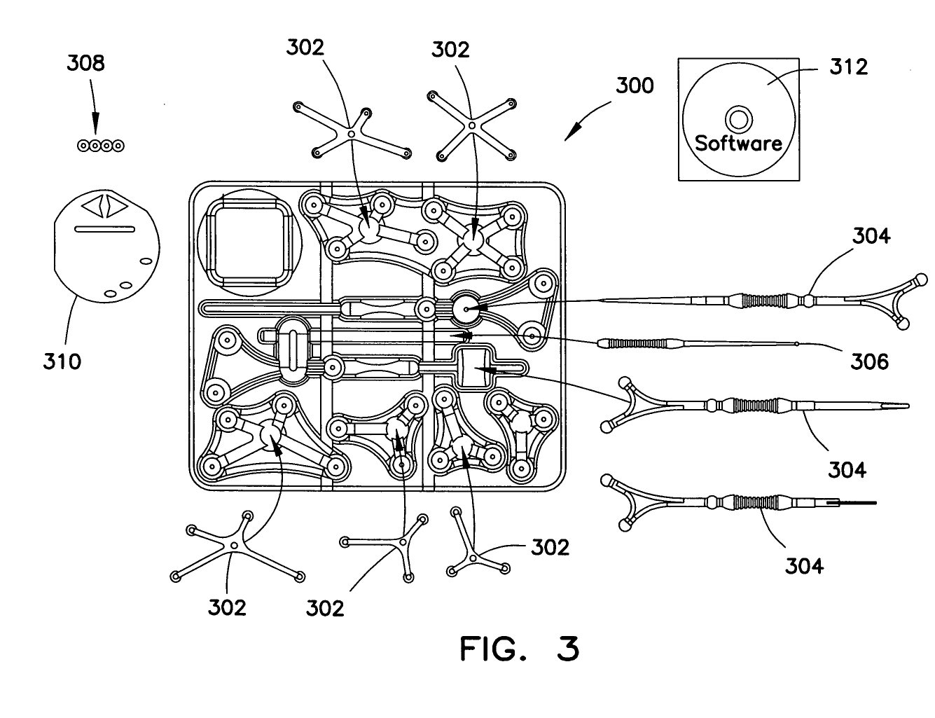

FIG. 3 shows orthopedic application kit 300, which is used in accordance with the present teachings. Application kit 300 is typically carried in a sterile bubble pack and is configured for a specific surgery. Exemplary kit 300 comprises arrays 302, surgical probes 304, stylus 306, markers 308, virtual keypad template 310, and application program 312. Orthopedic application kits are available for unicondylar knee, total knee, total hip, spine, and external fixation from EBI, L.P.

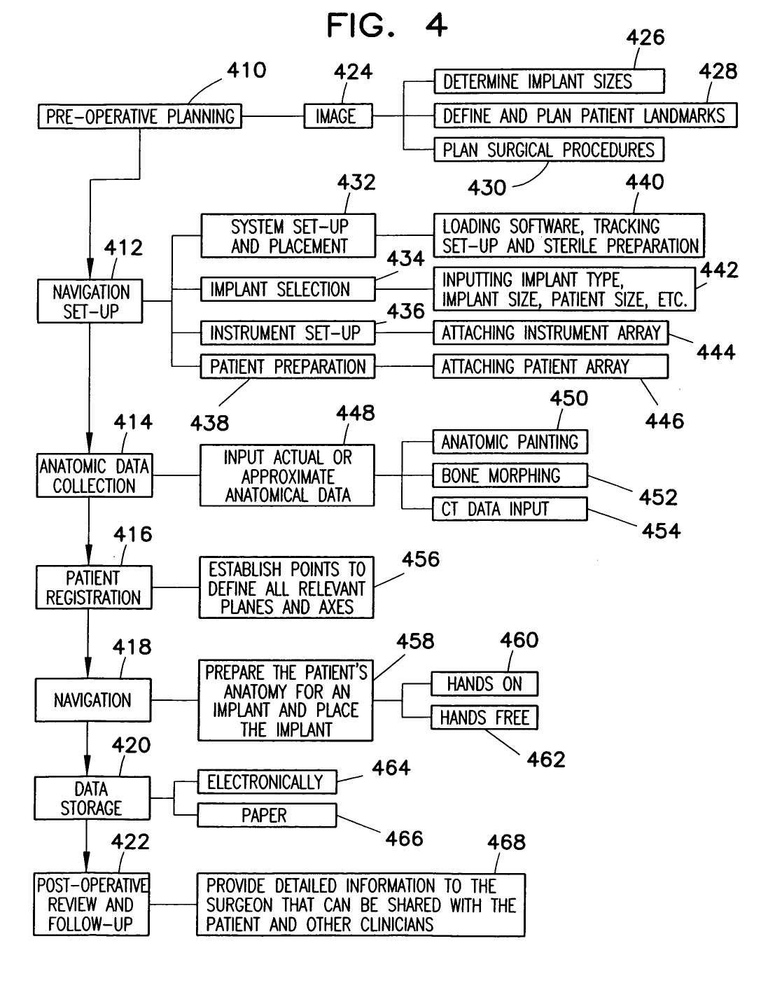

FIG. 4 shows an exemplary illustration of surgical navigation system 20. The process of surgical navigation according to this exemplary embodiment includes pre-operative planning 410, navigation set-up 412, anatomic data collection 414, patient registration 416, navigation 418, data storage 420, and post-operative review and follow-up 422.

Pre-operative planning 410 is performed by generating an image 424, such as a CT scan that is imported into the computer. With image 424 of the patient's anatomy, the surgeon can then determine implant sizes 426, such as screw lengths, define and plan patient landmarks 428, such as long leg mechanical axis, and plan surgical procedures 430, such as bone resections and the like. Pre-operative planning 410 can reduce the length of intra-operative planning thus reducing overall operating room time.

Navigation set-up 412 includes the tasks of system set-up and placement 432, implant selection 434, instrument set-up 436, and patient preparation 438. System set-up and placement 432 includes loading software, tracking set-up, and sterile preparation 440. Software can be loaded from a pre-installed application residing in memory, a single use software disk, or from a remote location using connectivity such as the internet. A single use software disk contains an application that will be used for a specific patient and procedure that can be configured to time-out and become inoperative after period of time to reduce the risk that the single use software will be used for someone other than the intended patient. The single use software disk can store information that is specific to a patient and procedure that can be reviewed at a later time. Tracking set-up involves connecting all cords and placement of the computer, camera, and imaging device in the operating room. Sterile preparation involves placing sterile plastic on selected parts of the surgical navigation system and imaging equipment just before the equipment is moved into a sterile environment, so the equipment can be used in the sterile field without contaminating the sterile field.

Navigation set-up 412 is completed with implant selection 434, instrument set-up 436, and patient preparation 438. Implant selection 434 involves inputting into the system information such as implant type, implant size, patient size, and the like 442. Instrument set-up 436 involves attaching an instrument array to each instrument intended to be used and then calibrating each instrument 444. Instrument arrays should be placed on instruments, so the instrument array can be acquired by the tracking system during the procedure. Patient preparation 438 is similar to instrument set-up because an array is typically rigidly attached to the patient's anatomy 446. Reference arrays do not require calibration but should be positioned so the reference array can be acquired by the tracking system during the procedure.

A anatomic data collection 414 involves a clinician inputting into the surgical navigation system actual or approximate anatomical data 448. Anatomical data can be obtained through techniques such as anatomic painting 450, bone morphing 452, CT data input 454, and other inputs, such as ultrasound and fluoroscope and other imaging systems. The navigation system can construct a bone model with the input data. The model can be a three-dimensional model or two-dimensional pictures that are coordinated in a three-dimensional space. Anatomical painting 450 allows a surgeon to collect multiple points in different areas of the exposed anatomy. The navigation system can use the set of points to construct an approximate three-dimensional model of the bone. The navigation system can use a CT scan done pre-operatively to construct an actual model of the bone. Fluoroscopy uses two-dimensional images of the actual bone that are coordinated in a three-dimensional space. The coordination allows the navigation system to accurately display the location of an instrument that is being tracked in two separate views. Image coordination is accomplished through a registration phantom that is placed on the image intensifier of the C-arm during the acquisition of images. The registration phantom is a tracked device that contains imbedded radio-opaque spheres. The spheres have varying diameters and reside on two separate planes. When an image is taken, the fluoroscope transfers the image to the navigation system. Included in each image are the imbedded spheres. Based on previous calibration, the navigation system is able to coordinate related anterior and posterior view and coordinate related medial and lateral views. The navigation system can also compensate for scaling differences in the images.

Patient registration 416 establishes points that are used by the navigation system to define all relevant planes and axes 456. Patient registration 416 can be performed by using a probe array to acquire points, placing a software marker on a stored image, or automatically by software identifying anatomical structures on an image or cloud of points. Once registration is complete, the surgeon can identify the position of tracked instruments relative to tracked bones during the surgery. The navigation system enables a surgeon to interactively reposition tracked instruments to match planned positions and trajectories and assists the surgeon in navigating the patient's anatomy.

During the procedure, step-by-step instructions for performing the surgery in the application program are provided by a navigation process. Navigation 418 is the process a surgeon uses in conjunction with a tracked instrument or other tracked array to precisely prepare the patient's anatomy for an implant and to place the implant 458. Navigation 418 can be performed hands-on 460 or hands-free 462. However navigation 418 is performed, there is usually some form of feedback provided to the clinician such as audio feedback or visual feedback or a combination of feedback forms. Positive feedback can be provided in instances such as when a desired point is reached, and negative feedback can be provided in instances such as when a surgeon has moved outside a predetermined parameter. Hands-free 462 navigation involves manipulating the software through gesture control, tool recognition, virtual keypad and the like. Hands-free 462 is done to avoid leaving the sterile field, so it may not be necessary to assign a clinician to operate the computer outside the sterile field.

Data storage 420 can be performed electronically 464 or on paper 466, so information used and developed during the process of surgical navigation can be stored. The stored information can be used for a wide variety of purposes such as monitoring patient recover and potentially for future patient revisions. The stored data can also be used by institutions performing clinical studies.

Post-operative review and follow-up 422 is typically the final stage in a procedure. As it relates to navigation, the surgeon now has detailed information that he can share with the patient or other clinicians 468.

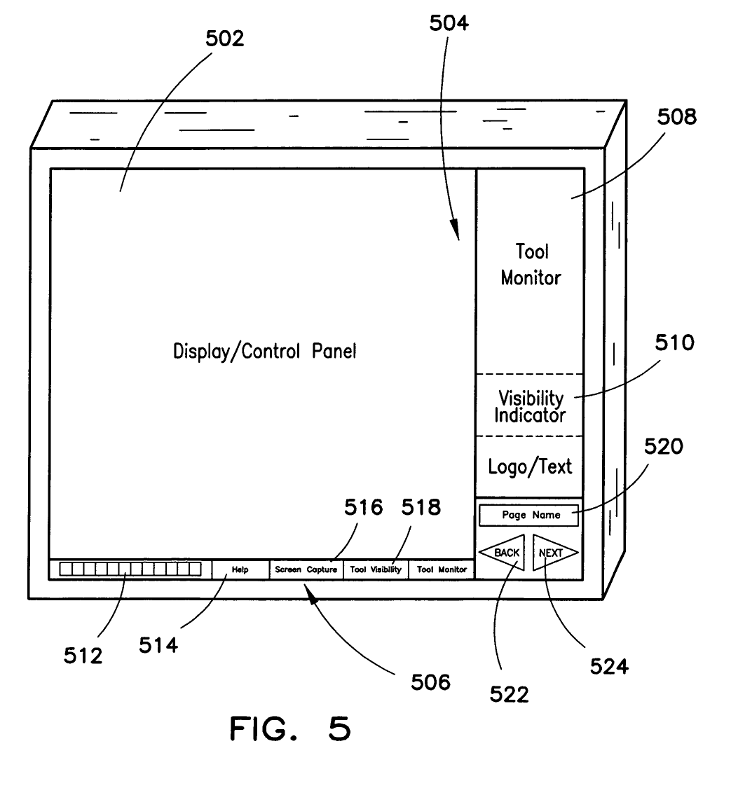

FIG. 5 shows a computer display layout embodiment in accordance with the present invention. The display layout can be used as a guide to create common display topography for use with various embodiments of input devices 114 and to produce visual outputs at output device 116 for core surgical navigation utilities 130, application programs 132, stored images 134, and application data 136 embodiments. Each application program 132 is typically arranged into sequential pages of surgical protocol that are configured according to a graphic user interface scheme. The graphic user interface can be configured with a main display 502, a main control panel 504, and a tool bar 506. The main display 502 presents images such as selection buttons, image viewers, and the like. The main control panel 504 can be configured to provide information such as a tool monitor 508, visibility indicator 510, and the like. The tool bar 506 can be configured with a status indicator 512, help button 514, screen capture button 516, tool visibility button 518, current page button 520, back button 522, forward button 524, and the like. The status indicator 512 provides a visual indication that a task has been completed, visual indication that a task must be completed, and the like. The help button 514 initiates a pop-up window containing page instructions. The screen capture button 516 initiates a screen capture of the current page, and tracked elements will display when the screen capture is taken. The tool visibility button 518 initiates a visibility indicator pop-up window or adds a tri-planar tool monitor to the control panel 504 above the current page button 520. The current page button 520 can display the name of the current page and initiate a jump-to menu when pressed. The forward button 524 advances the application to the next page. The back button 522 returns the application to the previous page. The content in the pop-up will be different for each page.

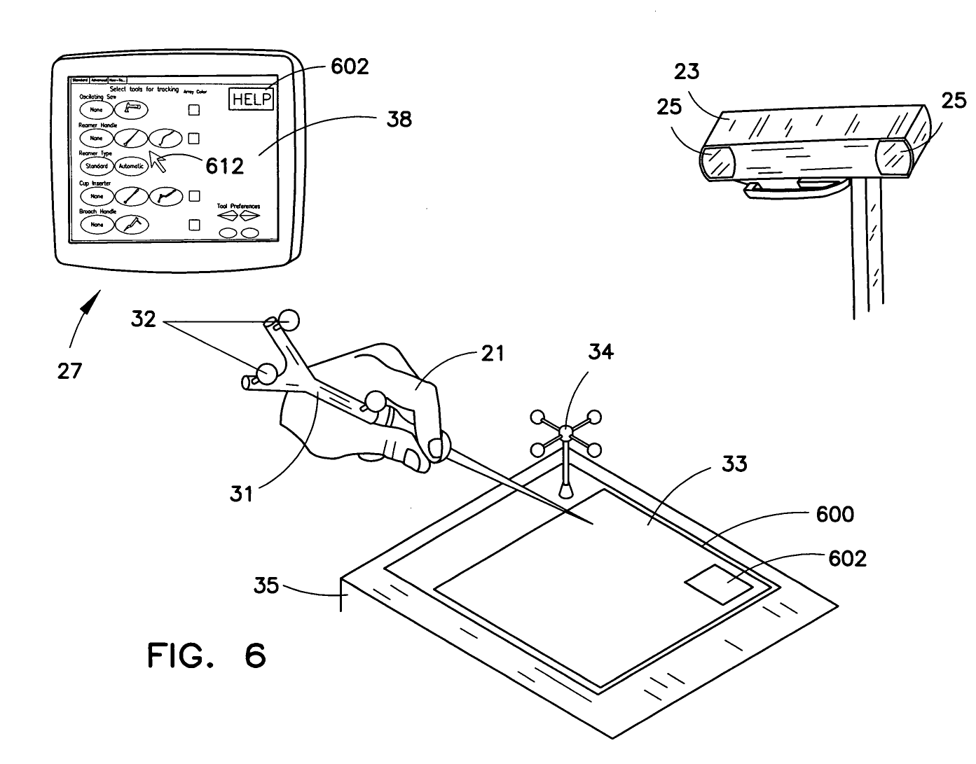

FIG. 6 illustrates a fragmentary perspective view of a virtual mouse in accordance of the present teachings as used in, e.g., part of an image guided hip procedure. The virtual mouse includes probe 31, pad or “touch pad” 33 and pad array 34. The probe includes three reflective spheres 32 that form a probe array. It is common to those of skill in this art to refer to the combination of probe 31 and spheres 32 as a “probe array,” and such reference is made occasionally herein. The touch pad includes a substantially flat surface as shown so that the tip of the probe can move along it, as described in further detail below.

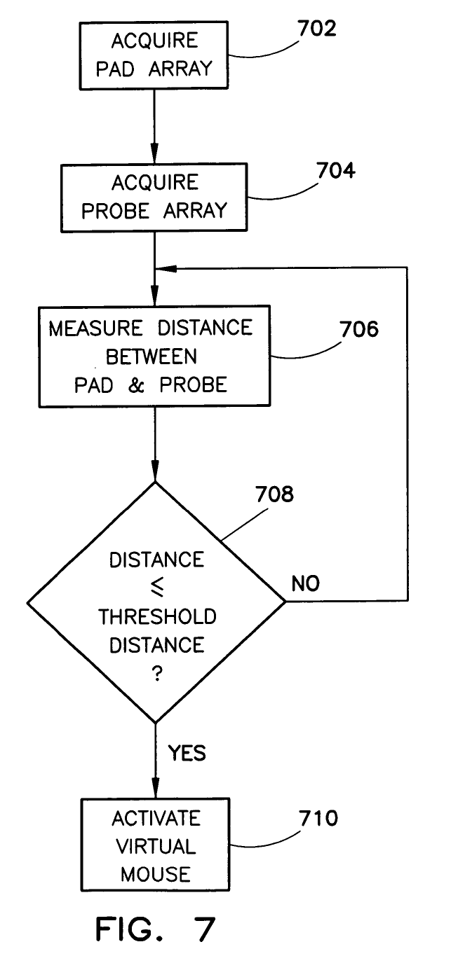

Activation of the virtual mouse is represented in the block diagram of FIG. 7. After the probe and touch pad are placed in the sterile field, surgical navigation system 20 must acquire them as shown in steps 702 and 704. These arrays are then tracked by the navigation system and the distance between them is calculated. Referring again to FIG. 6, the physician 21 points the probe 31 to the pad 33 that is supported by the table 35. The locating array 34 is used by the optical locator 23 to ascertain the location of the pad 33. By knowing the location of the pad 33 within the optical field, the location of the probe 31 can be tracked with respect to it. In the illustrated embodiment, the distance between the tip of probe 31 and the flat surface of touch pad 33 is determined, as depicted in step 706 of FIG. 7. The navigation system is programmed to activate the virtual mouse functionality when the probe 31 is positioned in close proximity to pad 33, as illustrated in blocks 708 and 710. The distance between the probe and pad at which the virtual mouse is activated is a design variable, but preferably is a few to several centimeters.

While physician 21 is preparing for or performing a surgery, the physician may select from a variety of icons shown in the computer display image 38 of the display 27 by using the virtual mouse functionality. Because the optical locator 23 senses the location of the probe 31 through use of the spheres 32, the location of the tip of the surgical probe 31 may also be determined. For instance, in FIG. 6, the tip of probe 31 is shown on display 38 as arrow 612 that is positioned close to the reamer handle icon. Those of skill in the art may interchangeably refer to arrow 612 as a “marker” or a “pointer,” and occasional reference to these alternate terms is made herein. By moving probe 31 with respect to pad 33, physician 21 correspondingly makes a mouse input, namely, moving arrow 612 on display 38.

While a “probe” is the preferred instrument to use with the virtual mouse due to the ability of its point to be precisely located, one of ordinary skill in the art would readily appreciate that surgical instruments other than a known probe or “probe array” could be substituted. Examples include spatulas, hook probes and similar instruments. Whatever instrument is used as the probe, it should have a tip and an array that allows it to be tracked by the navigation system.

The pad 33 can include a variety of indicia or “pad markers” to help the surgeon 21 navigate through the various icons on the computer display 38. For instance, the pad 33 can include a boundary or outline 600 which corresponds to a boundary or outline 602 of the computer display image 38. The boundary or outline 600 may be a visual indicator which is formed by paint, tape, or some other means of visual indication. The boundary 600 may also include a physical boundary such as a groove depression, or raised line such that the physician 21 may find the boundaries by touch when the probe crosses the physical features. In addition, the pad 33 also includes a help indicia 602, formed by either visual or physical indicators, such that the physician may select a help feature when desired. Furthermore, the pad may include indicia of a user interface screen.

While it is possible to include other indicia on the pad 33, typically only indicia corresponding to an icon on the computer display image which does not change from one image to another are displayed. It is within the scope of the teachings, however, to use a pad 33 which does not have any indicia including the boundary 600 or the help indicia 602. For instance, since the location of the probe 31 (determined by the markers 32) relative to the array 34, provides the required location data to the computer 12 to enable selection of the icons on the image 38.

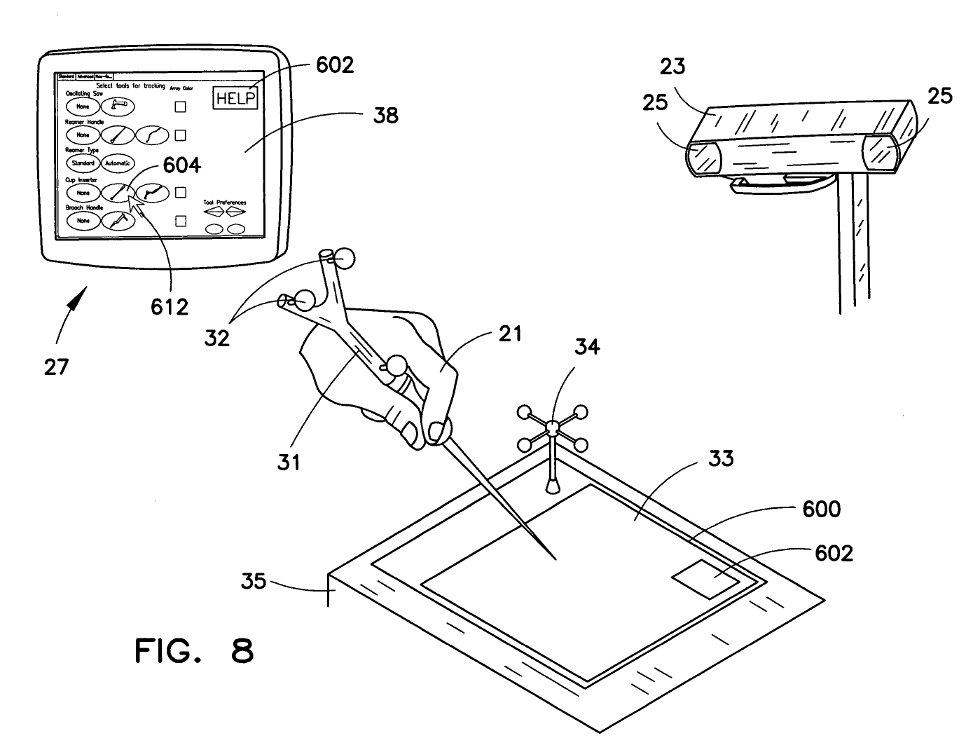

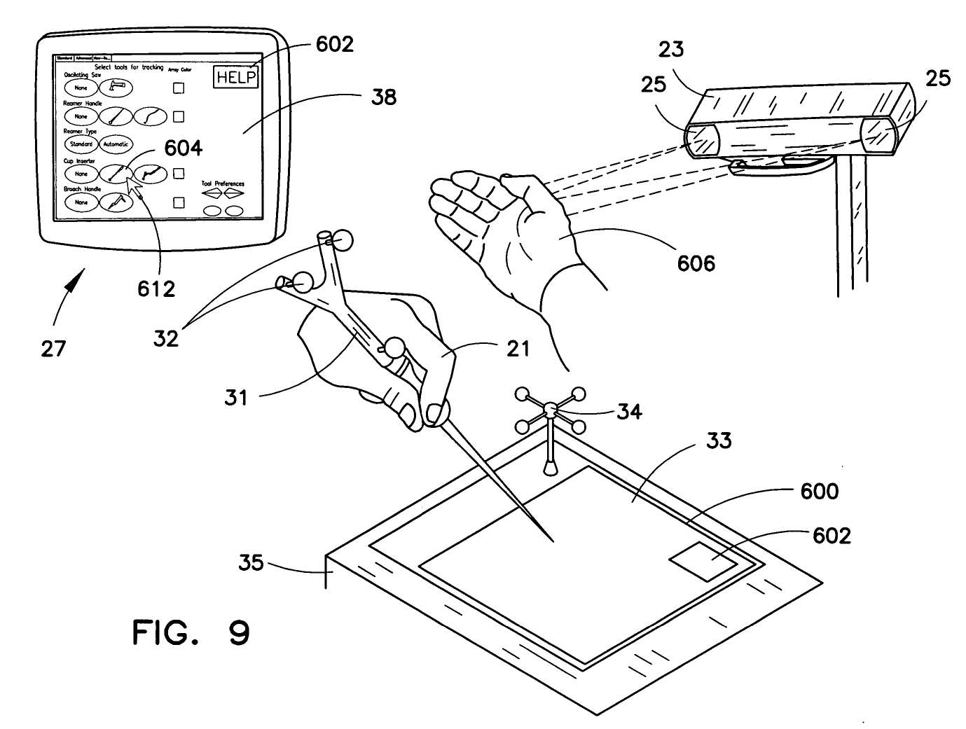

In FIG. 8, when the physician 21 has reached a point in the procedure where a cup inserter is required, the physician 21 moves the probe 31 to move pointer 612 to the icon 604 displayed on the computer display image 38. At this point in the procedure, the physician must select the icon 604 to move to the next page of the surgical protocol. To select the icon 604, the physician 21, as illustrated in FIG. 9, occludes or blocks the markers 32. The markers 32 may be occluded or blocked with the physician's free hand 606 or by other means. The break in the optical path between the markers 32 and cameras 25 is recognized by the computer 112. Once the markers are no longer sensed, the computer system indicates to the physician 21 that the icon 604 has been selected by changing the appearance of the icon. For instance, the color of the icon may be changed. It is also within the scope of the present teachings to indicate the selection of the icon 604 by other means or methods such as flashing the icon on and off or increasing the brightness of the icon 604. In addition, the screen 38 may include an indicator for the physician which provides information regarding how long the optical path should be blocked to select the icon 604. Once the physician decides to select the icon, the physician 21 removes his free hand 606 from the optical path. At this point, the computer system recognizes the re-establishment of the optical path to the markers 32 which causes the computer system to proceed to the next computer display image 38.

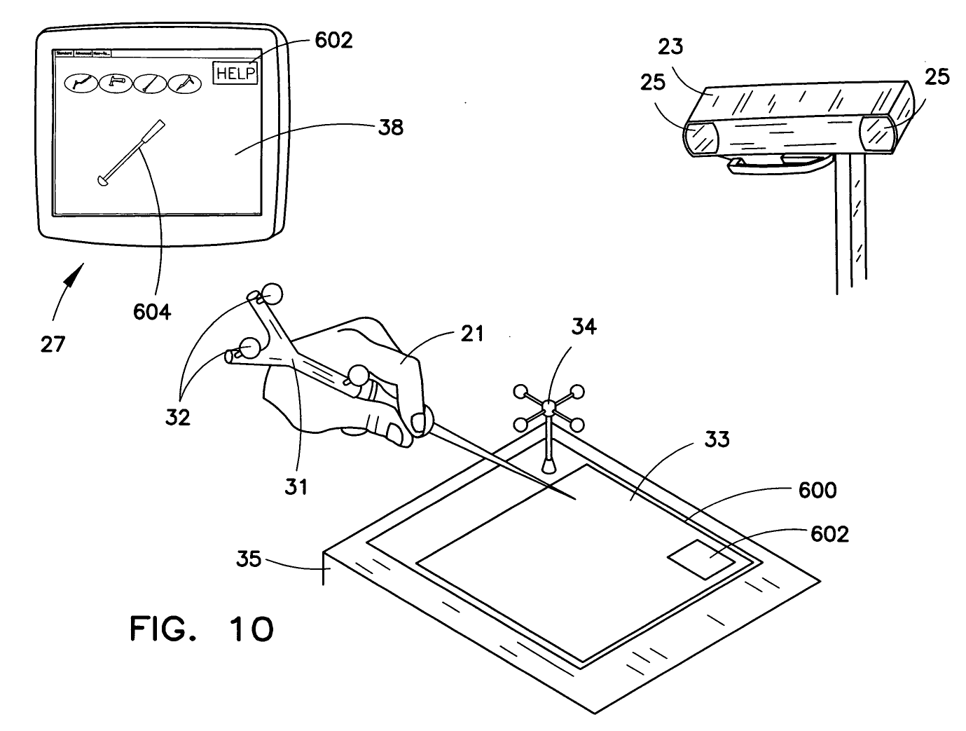

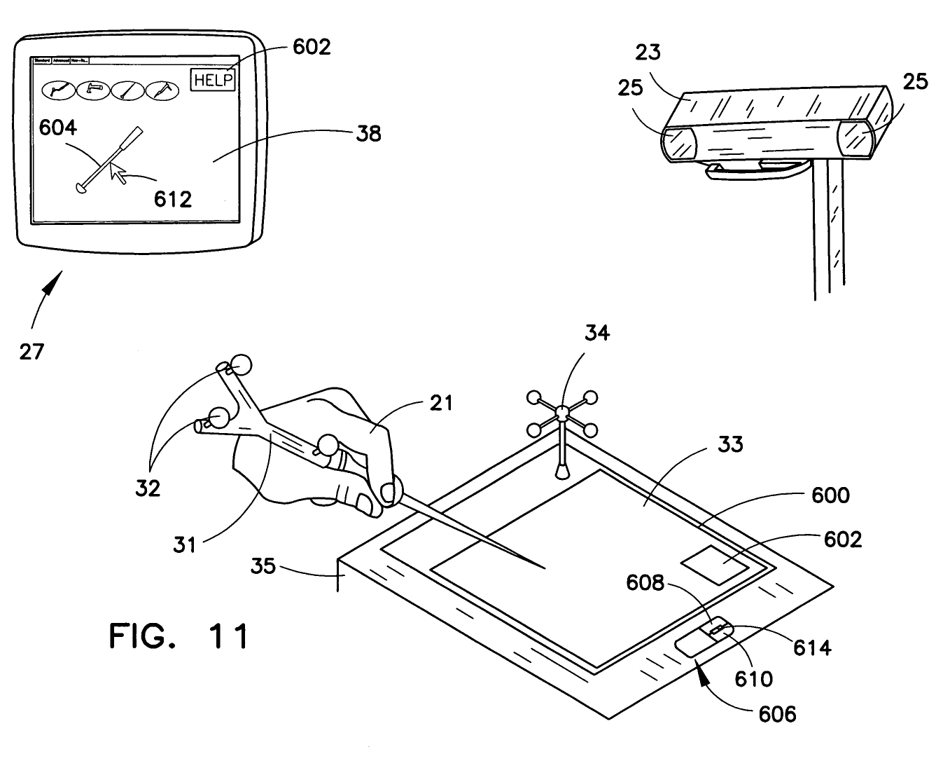

Referring now to FIG. 10, the next selected page of surgical protocol is shown which illustrates a more detailed display of the cup inserter 604. Once the cup inserter display 604 has been selected, the physician 21 can put down the surgical probe 31 and pick up the cup inserter so that the cup inserter may be appropriately identified or registered by the computer system.

As described with respect to FIG. 9, selective gesturing by occlusion of the optical path 606 makes a virtual mouse input, in this case, selecting an icon. As previously described, occluding the optical path for a certain period of time may be recognized by the computer as being equivalent to a click of a left mouse button on a conventional computer mouse. It is also within the scope of the present teachings to perform a double click on a button by occluding the optical path for a period of time, unblocking the optical path for a period of time, blocking the optical path again for a period of time and then unblocking the optical path. For a further description of selective gesturing, see U.S. Provisional Patent Application Ser. No. 60/693,461, titled “Selective Gesturing Input to a Surgical Navigation System” (hereinafter “Selective Gesturing application”), filed Jun. 23, 2005, which is incorporated by reference herein in its entirety.

In a further embodiment, as illustrated in FIG. 1, the table 35 may include an image or replica of a mouse (or mouse) 606. It is also within the scope of the present teachings to include the image 606 within the boundary 600. The image 606 includes a left mouse button 608 and a right mouse button 610. To select the icon 604, the physician may move the probe 31 to point the pointer 612 to the icon 604 first, block the optical path to make a new selection, and then move the pointer 612 to the left mouse button 608 or right mouse button 610 to thereby use the known features of a mouse as is understood by those skilled in the art. For instance, selecting the mouse button 608 may be used to select an icon or a menu item. A double click or button 608 by using occlusion as previously described may provide for opening the next screen relating to an icon. Likewise, the right mouse button 610 may be used to bring up a menu of available selections. Consequently, it is within the scope of the present teachings to incorporate all of the known features of a mouse button or buttons including a selector wheel 614. Consequently, these teachings provide the function of a virtual mouse for enabling a physician 21 or technician to select various icons which are displayed on the display screen 38 and to move from one display screen to another without leaving the sterile field.

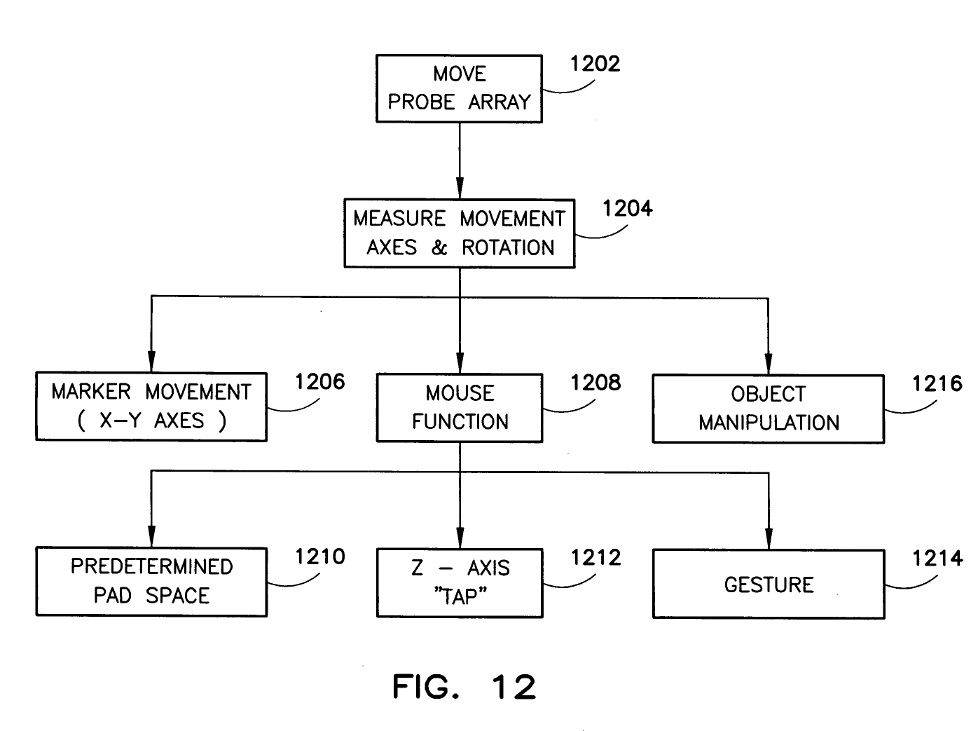

Having described a specific example employing the virtual mouse of the present teachings, a more generalized block diagram representing the virtual mouse functionality can be appreciated. As shown in FIG. 12, movement of probe array 31 (block 1202) is measured (block 1204). There are multiple types of movement that result in different mouse functionality or mouse inputs. For example, in block 1206, planar movement (x-y axes) of the probe along the surface of pad 33 is recognized by the system and correspondingly moves the arrow or marker on the screen, as described above. This is typically the mouse input that is used most.

Block 1208 represents mouse functionality that is further broken down in blocks 1210, 1212 and 1214 into “predetermined pad space,” “z-axis” and “gesture,” respectively. As described above with reference to FIG. 10, one example of a predetermined pad space is replica 606 that includes indicia of left and right mouse buttons and a scroll button. In these predetermined pad spaces (unlike the major surfaces of pad 33), the system recognizes movement of the probe as corresponding to a specific mouse function that is typically different than merely moving the arrow or marker on the monitor. For example, the predetermined space may include a pad marker indicia of a scroll dial, which, when the tip of the probe is moved along it, causes the monitor to scroll.

The system may also recognize and assign functionality to movement of the tip of the probe away from the surface of the pad, i.e., along the z-axis, as shown at block 1212. For example, a quick movement of the tip of the probe away from the pad a few centimeters and then returning the tip to substantially the same spot on the pad may be interpreted as equivalent to a single click of a conventional mouse. Similarly, two of these short “taps” may be interpreted as a double click. One of skill in the art would readily recognize many other functions or mouse inputs that could be assigned to various movements of the probe in the z-axis.

As described above with reference to FIG. 8, mouse functionality can be obtained through gesturing as indicated in block 1214. The gesturing can be interpreted by the system as equivalent to the click of a conventional mouse, or can be interpreted as other functions, such as equivalent to a “right click” of a conventional mouse. One of skill in the art would readily recognize many other functions that could be assigned to gesturing of the probe or pad arrays. A detailed description of selective gesturing is provided in the Selective Gesturing application incorporated by reference above.

These teachings also provide “object manipulation” capabilities (block 1216). For example, the tip of the probe may be moved across the flat surface of the pad, which causes corresponding movement of the pointer or arrow on the monitor, as described elsewhere. The arrow is moved until it is positioned over an image of human anatomy, such as a knee, for example. The probe may then be lifted from the flat surface of the pad, which is recognized by the computer as a mouse input triggering “object manipulation” mode. Once in this object manipulation mode, the computer translates three dimension movement of the probe to corresponding three dimensional movement of the image on the monitor. While the exact correspondence between the three-dimensional movement of the probe and movement of the image is a design variable, it is preferable that the correspondence be intuitive. For example, rotating the probe along its long axis would rotate the image of the knee about the axis of the bone.

While an exemplary embodiment incorporating the principles of the present invention has been disclosed hereinabove, the present invention is not limited to the disclosed embodiments. Instead, this application is intended to cover any variations, uses, or adaptations of the invention using its general principles. For instance, instead of providing a pad, the present invention may include a stand marked appropriately and including an array 34. Further, this application is intended to cover such departures from the present disclosure as come within known or customary practice in the art to which this invention pertains and which fall within the limits of the appended claims.

What is claimed is:

1. A method of performing a surgery, comprising:
operating a surgical navigation system having a tracking system, computer and monitor placed outside of a sterile field;
placing a pad having a pad array within the sterile field;
placing a probe having a probe array within the sterile field;
acquiring the pad array and the probe array with the tracking system;
activating a virtual mouse by moving the probe near the pad; and
making a mouse input to the computer with the virtual mouse.

2. The method of claim 1, wherein the mouse input comprises moving a pointer on the monitor.

3. The method of claim 1, wherein the probe is moved along a substantially flat surface of the pad to make the mouse input.

4. The method of claim 3, further comprising moving the probe away from the surface to make a second mouse input to the computer.

5. The method of claim 4, wherein the second mouse input comprises selecting a function.

6. The method of claim 1, further comprising occluding the probe array to make a second mouse input to the computer.

7. The method of claim 1, wherein the probe is moved to a pad marker disposed on the pad to make a second mouse input.

8. The method of claim 1, wherein the probe is moved in three dimensions to manipulate a corresponding object on the computer monitor.

9. The method in claim 8, wherein the corresponding object is a human anatomy image.

10. A surgical navigation system, comprising:
a computer having surgical navigation utilities software;
a tracking system coupled to the computer for recognizing and tracking movement of arrays within a measurement field;
a monitor coupled to the computer;
a virtual mouse input device, including,
a pad having a pad array, the pad array being trackable by the tracking system and recognizable by the computer as associated with the pad; and
a probe having a probe array, the probe array being trackable by the tracking system and recognizable by the computer as associated with the probe;
wherein movement of the probe relative to the pad causes a mouse input to the computer.

11. The surgical navigation system of claim 10, wherein the mouse input comprises movement of a pointer on the monitor.

12. The surgical navigation system of claim 10, wherein the pad comprises a pad marker which is configured to cooperate with the probe to provide a second mouse input to the computer.

13. The surgical navigation system of claim 10, wherein the pad has a substantially flat surface and the probe has a tip which when moved along the substantially flat surface is configured to cause the mouse input.

14. The surgical navigation system of claim 13, wherein the mouse input comprises movement of a pointer on the monitor.

15. A virtual mouse input device for a surgical navigation system, comprising:
a touch pad adapted for use in a sterile field;
a pad array attached to the touch pad, the pad array being trackable by a surgical navigation system and recognizable by the surgical navigation system as a touch pad; and
a probe suitable for use in a sterile field;
a probe array attached to the probe, the probe array being trackable by the surgical navigation system in relation to the pad array;
wherein movement of the probe array corresponds to movement of a marker on a monitor coupled to the surgical navigation system.

16. The virtual mouse of claim 15, wherein the touch pad has a substantially flat surface and the probe has a tip configured to move along the substantially flat surface to cause the movement of the marker on the monitor.
